(19) United States
(12) Patent Application Publication
(10) Pub. No.: US 2018/0080860 A1
(43) Pub. Date: Mar. 22, 2018

(54) METHOD FOR DENSITY MEASUREMENT USING MULTIPLE SENSORS

(71) Applicant: UOP LLC, Des Plaines, IL (US)

(72) Inventors: Robert Roemisch, Des Plaines, IL (US); Nelson Wayne Lytle, Bolingbrook, IL (US); Joseph Heetel, Riverside, IL (US)

(21) Appl. No.: 15/649,972

(22) Filed: Jul. 14, 2017

Related U.S. Application Data
(60) Provisional application No. 62/367,484, filed on Jul. 27, 2016.

(51) Int. Cl. G01N 9/00; G01N 33/28

(52) U.S. Cl. CPC G01N 9/00; G01N 33/2823

(57) ABSTRACT

A process for on-line density measurement for a hydrocarbon fluid. The hydrocarbon fluid is caused to flow through first and second density sensors arranged in series. The first density sensor has a first temperature and the second density sensor has a second temperature, defining a temperature difference between the first temperature and the second temperature. A first density measurement and second density measurement are received from the first and second density sensors, respectively. A temperature conversion factor is determined. The first density measurement or the second density measurement is corrected using the determined temperature correction factor to provide a temperature-corrected density measurement.

### CROSS-REFERENCE TO RELATED APPLICATION

This application claims priority from Provisional Application No. 62/367,484 filed Jul. 27, 2016, the contents of which cited application are hereby incorporated by reference in its entirety.

### FIELD OF THE INVENTION

This invention relates generally to methods and systems for measuring fluid properties.

### BACKGROUND OF THE INVENTION

It is desirable to provide on-line measurement of density or density-related properties (collectively, density measurement) of a hydrocarbon fluid in a hydrocarbon processing environment, such as a plant or laboratory. Examples of density-related properties include specific gravity and American Petroleum Institute (API) gravity. “On-line” refers to density measurement of a hydrocarbon fluid, e.g., density measurement sampling that occurs during operation of a hydrocarbon process.

On-line density measurement sampling has a higher resolution than, for instance, sampling the hydrocarbon fluid's weight once per day, which can miss problems in a hydrocarbon processing environment that an on-line density measurement can catch. Such problems include downward cycling due to loss of temperature control in a vacuum column affecting an overhead fraction. On-line density measurement allows tighter control of hydrocarbon processes such as hydrocracking processes, improves throughput, saves operating costs by minimizing plant line out time, and saves in laboratory analysis costs.

A concern with current on-line density measurement methods is converting from operating temperatures in hydrocarbon processing environments, such as plant or laboratory operating temperatures to standard temperatures used in density calculations (standard temperature conversions). The conversion factor for standard temperature conversions depends on the fluid composition, and thus will change as the fluid composition changes. Some density measurements use American Society for Testing and Materials (ASTM) Petroleum Conversion tables to perform standard temperature conversions, but such methods are not amenable to on-line determination.

There is a need in the art for a method to convert on-line density measurements of a hydrocarbon fluid to standard temperature in real-time for monitoring hydrocarbon conversion and process control.

### SUMMARY OF THE INVENTION

The present invention is directed to providing effective and efficient processes for on-line measurement of density of a hydrocarbon fluid in a hydrocarbon processing environment.

Accordingly, in one aspect of the present invention, the present invention provide method for on-line density measurement for a hydrocarbon fluid. The hydrocarbon fluid is caused to flow through first and second density sensors arranged in series, the first density sensor having a first temperature and the second density sensor having a second temperature, wherein a temperature difference of between 5° C. and 100° C. is defined between the first temperature and the second temperature. A first density measurement is received from the first density sensor, and a second density measurement is received from the second density sensor. A temperature conversion factor is determined using the first density measurement, the second density measurement, the first temperature, and the second temperature. Either the first density measurement or the second density measurement is corrected using the determined temperature correction factor to provide a temperature-corrected density measurement for the hydrocarbon fluid.

Additional objects, embodiments, and details of the invention are set forth in the following detailed description of the invention.

### DETAILED DESCRIPTION OF THE DRAWINGS

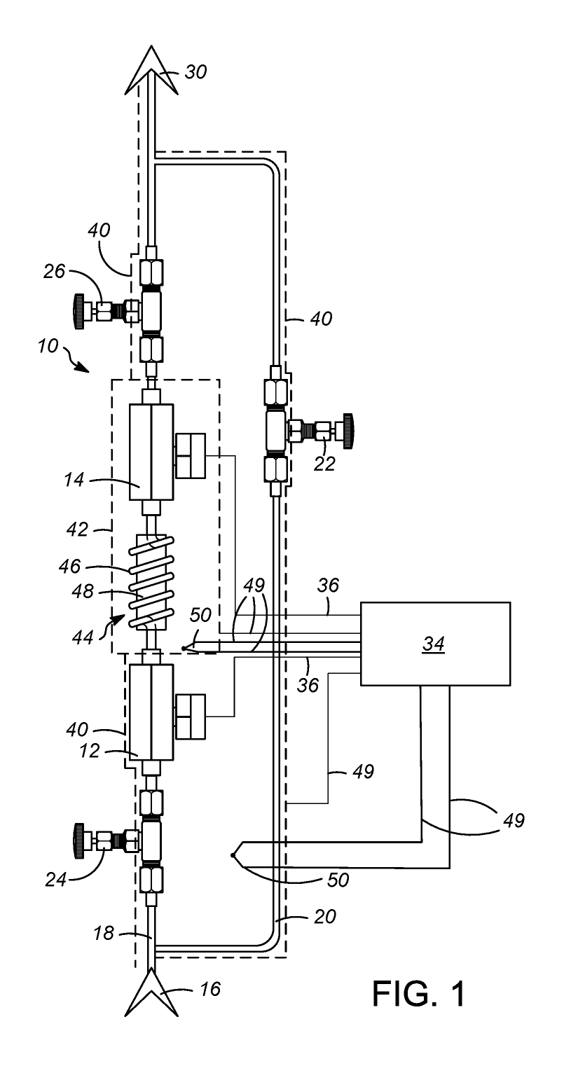

FIG. 1 shows an example apparatus for on-line density measurement of a hydrocarbon fluid; and,

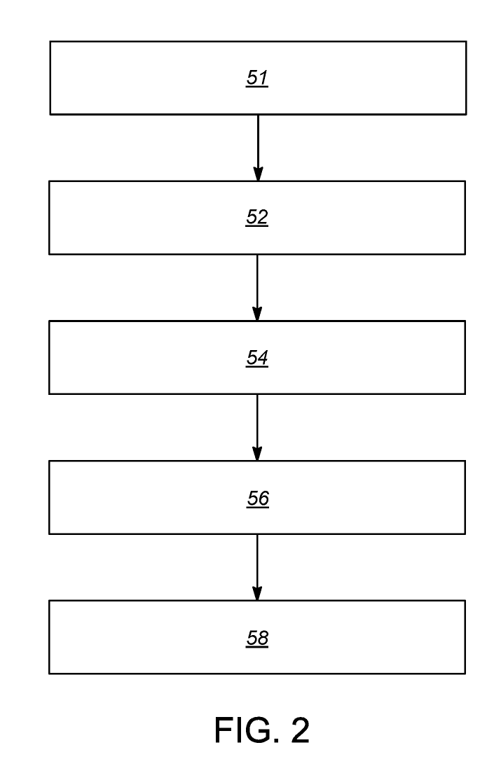

FIG. 2 shows an example process for on-line density measurement of a hydrocarbon fluid.

### DETAILED DESCRIPTION OF THE INVENTION

Generally, in an example method to perform on-line density measurement of a hydrocarbon fluid, the hydrocarbon fluid is caused to flow through first and second density sensors that are arranged in series to provide a sensor array. The first and second density sensors are maintained at first and second temperatures. The first and second temperatures are predetermined and distinct, creating a predefined non-zero temperature difference, or delta temperature, between the first and second density sensors. This temperature difference is preferably between 5° C. and 100° C. (considering properties of the hydrocarbon fluid at the resulting temperature), more preferably between 7° C. and 50° C., and most preferably between 10° C. and 25° C. The first temperature may be a higher temperature relative to the second temperature, and the second temperature may thus be a relatively lower temperature relative to the first temperature. Alternatively, the second temperature may be a higher temperature relative to the first temperature, and the first temperature may thus be a relatively lower temperature relative to the second temperature. The difference between the first temperature and the second temperature provides the delta temperature.

The first and second density sensors produce first and second density measurements, respectively, for the hydrocarbon fluid. A processor can receive the first and second density measurements, and with knowledge of the first and second temperatures, can determine (e.g., calculate) a temperature conversion factor, such as a density temperature coefficient, or a density-related coefficient such as a specific gravity (SG) or API coefficient, on the fly. For example, an equation describing a temperature effect on density is provided in Equation (1) below:

$${D}_{1} = \frac{{D}_{0}}{( 1 + B ( {T}_{1} - {T}_{0} ) )} \qquad ( 1 )$$

where D₁ is the density at a process temperature, D₀ is the standard temperature, B is the temperature coefficient, T₁ is the process temperature, and T₀ is the standard temperature.

The B coefficient in Equation (1) is strongly affected by the composition of the fluid. Since fluid composition changes are commonplace in hydrocarbon processing operations, e.g., feed and conversion changes in pilot plant operations, it is useful to calculate the B coefficient on the fly.

In an example method, the B coefficient can be solved using Equation (1) by inputting the first and second densities for D₀ and D₁ as measured in the sensor array on-line, and inputting the associated first and second temperatures for T₀ and T₁. Once the temperature conversion factor, such as the B coefficient, is determined, the same relationship in Equation (1) can then be used to convert the density measured from either the first or the second density sensor to a temperature-corrected density measurement, such as the density at standard temperature. Since the first and second density measurements are performed on-line, the calculated temperature conversion factor reflects the instantaneous composition of the hydrocarbon fluid. This eliminates the need to use tables to calculate a corrected density measurement or corrected density-related measurement (SG/API).

Referring now to the drawings, FIG. 1 shows an example density measurement apparatus 10 for density measurement of a hydrocarbon fluid. The density measurement apparatus 10 includes first and second density sensors 12, 14 arranged in series, providing a dual sensor array. However, it is contemplated that the density measurement apparatus 10 could include more than two density sensors. The density sensors 12, 14 are preferably embodied in fluid properties sensors, a particular example of which is FPS2800 fluid properties sensor, manufactured by Measurement Specialties, Hampton, Va.

The density measurement apparatus 10 can be installed into a fluid line in a hydrocarbon fluid processing system, such as a plant and/or laboratory using a double block and bleed configuration. The fluid line may be, for instance, a fluid line for a vacuum overhead stream, a vacuum bottoms stream, an atmospheric overhead stream, or for other streams. As a more particular example, the fluid line may be for a vacuum bottoms stream between a vacuum distillation column level control valve and a product collection. As another particular example, the fluid line may be for a vacuum overhead stream in a recycle loop prior to a level control valve.

To install the density measurement apparatus 10 into the fluid line, an inlet 16 of the density measurement apparatus 10 is coupled to a portion of the fluid line (not shown) of the hydrocarbon fluid processing system using a suitable fluid coupling, e.g., a fitting such as those manufactured by SWAGELOK™, Solon, Ohio. The inlet 16 is in fluid communication with a first fluid line 18, in which the first and second density sensors are arranged in series, and an additional, second fluid line 20, which bypasses the first and second density sensors 12, 14. The second line 20 includes a valve 22 for controlling fluid flow through the second line, and the first line 18 includes a valve 24 disposed before the first density sensor 12 and another valve 26 disposed after the second density sensor 14 for controlling fluid flow through the first line. The first and second lines 18, 20 merge into an outlet 30, which can be coupled to another portion of the fluid line (not shown) of the hydrocarbon processing system, using suitable fittings, e.g., SWAGELOK™ fitting. The first and second lines 18, 20 provide a block and bleed configuration for the density measurement apparatus 10, which allows an easy change out of the first or the second density sensors 12, 14 if sensor maintenance is necessary, while allowing the hydrocarbon fluid process to continue to run. The first and second density sensors 12, 14 are installed in the first line 18 using suitable fittings, e.g., SWAGELOK™ fitting.

The first and second density sensors 12, 14 are coupled to a processor 34 via suitable signal lines 36 for receiving and processing data from the first and second density sensors. For example, the processor 34 can be embodied in or include a computer (e.g., a PC or other computer), a network of computers, an application specific integrated circuit (ASIC), a server, a client, a mobile device, or any suitable processing device or network of connected processing devices which include computer-readable instructions that when executed perform one or more steps of example methods described herein. Each of the signal lines 36 can be embodied in, for example, a signal bus, an Ethernet communication line, a wireless transmitter and receiver, or others, in any suitable combination. In a particular example, the signal lines 36 are embodied in controller area network (CAN) bus lines, which are coupled to a gateway via an Ethernet connection to adaptor boxes. The gateway is coupled to a processor embodied in a suitable computer (e.g., a CPU, PC, server, etc., including individual or multiple, networked computers) or other processor (e.g., programmed hardware, ASIC, etc., including individual or multiple, connected processors) for data collection and analysis.

The first and second density sensors 12, 14 are preferably maintained at first and second predetermined temperatures during operation of the density measurement apparatus 10 so that a predetermined non-zero delta temperature is defined between the first and second density sensors. This temperature difference is preferably between 5° C. and 100° C. (considering properties of the hydrocarbon fluid at the resulting temperatures), more preferably between 7° C. and 50° C., and most preferably between 10° C. and 25° C. For example, in the density measurement apparatus 10 shown in FIG. 1, the first density sensor 12 can be configured to be maintained at a lower temperature relative to the second density sensor 14, and thus the first density sensor can be considered a low temperature sensor. The second density sensor 14 is configured to be maintained at a higher temperature relative to the first density sensor 12, and thus the second density sensor can be considered a high temperature sensor. Here, “high” and “low” are with reference to relative temperatures. In an alternative embodiment, the first density sensor 12 can instead be a high temperature sensor and the second density sensor 14 a low temperature sensor.

In an example embodiment, to maintain the first and second density sensors 12, 14 at predetermined low and high temperatures, respectively, the density measurement apparatus 10, and particularly the first line 18 and the second line 20, are insulated and/or regulated. Insulation and/or regulation can include, for instance, wrapping the first line 18 and the second line 20 in heat tape or using insulation such as fiberglass wrapping, to provide an outer, lower temperature zone 40 (illustrated by dashed lines in FIG. 1) at a predetermined lower temperature. It is also contemplated that the lower temperature zone 40 could be selectively heated or cooled by an external heater or cooler to provide the predetermined lower temperature. The first density sensor 12 is disposed within the lower temperature zone 40.

A higher temperature zone 42 is disposed within the lower temperature zone 40, and the second density sensor 14 is disposed within higher temperature zone 42. Alternatively, the higher temperature zone 42 and the lower temperature zone 40 could be respectively contained within two separate temperature-controlled zones. Insulation and/or temperature regulating material, e.g., heat tape, fiberglass, etc. or other material, can fully or partially surround the higher temperature zone 42 to maintain a higher temperature. The higher temperature zone 42 can be selectively regulated through a controlled heater or cooler. In a particular example, the higher temperature zone 42 is embodied in a controlled heater (heater) having an internal cavity for receiving the second density sensor 14. Example heaters include heated fluids bled into the higher temperature zone 42, e.g., with valve feedback; electrical jacketing with thermal sensors; or steam jacketing with thermal sensors. A particular example heater for the higher temperature zone 42 is a GLASCOL™ heater, manufactured by Glas-Col of Terra Haute, Indiana, which includes a heating element for controlled heating of a fluid. This example heater includes a holder into which the second density sensor 14 can be disposed.

In an example embodiment, to alleviate temperature cycling within the sensor, a temperature stabilizer 44 is provided within the higher temperature zone 42 upstream of the second (high temperature) density sensor 14. The example temperature stabilizer 44 is embodied in an equilibration coil including a section of tubing 46, e.g., stainless steel tubing, wrapped around a cylinder 48 such as a stainless steel bar. The temperature stabilizer 44 physically delays the fluid entering the high temperature zone 42 before the fluid is measured by the second (higher temperature) density sensor 14. Another temperature stabilizer (not shown) such as an equilibration coil, can be provided in the first line 18 upstream of the first (low temperature) sensor 12 to condition the fluid before it enters the first sensor.

The heater or other temperature regulator for the higher temperature zone 42 can be controlled via the processor 34, or via another device coupled to or separate from the processor. The processor 34 may be coupled to the temperature regulator in the higher temperature zone 42, e.g., via suitable signal lines 49, to selectively control the temperature regulator. Similarly, if an additional temperature regulator is provided for controlling a temperature of the lower temperature zone 40, the temperature regulator can be controlled via the processor 34, e.g., coupled via signal lines 49, or via another device coupled to or separate from the processor. Temperature regulators may be controlled independently of the processor 34.

Temperature feedback for the higher and lower temperature zones 40, 42 can be provided by temperature sensors 50 such as but not limited to thermocouples or thermistors. The temperature sensors 50 may be disposed at or near the first and/or second density sensors 12, 14, disposed on the insulation or wrapped temperature regulator (e.g., sensors disposed on heat tape), and/or disposed at or integrated with the temperature regulator (e.g., thermocouples disposed in thermowells of a cell holder for the temperature regulator). The temperature sensors can be coupled to the processor 34 or other device via the signal lines 49 or other suitable signal paths for providing temperature feedback. Control of the temperature of the higher temperature zone 42 (or the lower temperature zone 40, if actively controlled) can be provided using such feedback, e.g., closed loop control.

Though, in the example density measurement apparatus 10, the lower temperature zone 40 is insulated to provide a lower temperature for the first density sensor 12, and the higher temperature zone 42 is heated to provide a higher temperature for the second density sensor 14, it will be appreciated that the temperature of the lower temperature zone and the higher temperature zone can be regulated in other ways. For example, the temperature of the lower temperature zone 40 can be provided by cooling, while the temperature of the higher temperature zone 42 is either maintained or raised. In general, each of the lower temperature zone 40 and the higher temperature zone 42 can be heated, maintained, or cooled such that that a predetermined, non-zero temperature difference can be defined between the lower temperature zone and the higher temperature zone, and thus between the first density sensor 12 and the second density sensor 14.

In a non-limiting example embodiment, a minimum of 20° C. is maintained as the temperature difference (delta temperature, or delta T) between the first density sensor 12 and the second density sensor 14 during operation of the density measurement apparatus 10. This can be provided, for instance, by maintaining the temperature difference between the lower temperature zone 40 and the higher temperature zone 42. This allows a sufficient resulting difference in density measurements to minimize errors when calculating the temperature conversion factor (e.g., the B coefficient in Equation (1) above). A wider delta T minimizes noise in on-line measurements for the first and second density sensors 12, 14, creating a more stable temperature conversion factor calculation, and thus more reliable temperature compensated density measurements. The delta T can also be selected based on the temperature of the process stream through the fluid line 18. For lighter fluid streams, a smaller delta T may be preferred, for instance to avoid creating bubbles in the stream. In a preferred embodiment, a temperature for the process stream may provide the lower temperature for the lower temperature zone 40, and the higher temperature can be chosen to be a temperature that provides the largest delta T, but that is less than the bubble point of the fluid. This temperature difference is preferably between 5° C. and 100° C. (considering properties of the hydrocarbon fluid at resulting temperatures), more preferably between 7° C. and 50° C., and most preferably between 10° C. and 25° C.

FIG. 2 shows an example process for on-line density measurement of a hydrocarbon fluid, which will be explained with reference to the example density measurement apparatus 10 shown in FIG. 1. The hydrocarbon fluid is caused to flow through the first line 18, and thus through the first and second density sensors 12, 14, e.g., by a pressure difference provided by a hydrocarbon processing system in which the density measurement apparatus 10 is installed, or by an additional pressure difference provided separately from the hydrocarbon processing system.

The first density sensor 12 is maintained at the first temperature (in the density measurement apparatus 10, a lower temperature), and the second density sensor 14 is maintained at the second temperature (e.g., a higher temperature), while the hydrocarbon fluid flows through the first density sensor and the second density sensor (step 51). For example, the lower temperature zone 40 can be maintained at the first, lower temperature, and the higher temperature zone 42 can be maintained at the higher temperature, as provided above.

In step 52, a lower temperature density D_{TLo} is measured from the first density sensor (lower temperature sensor) 12 and a higher temperature density D_{THi} is measured from the second density sensor (higher temperature sensor) 14. The measurements in step 52 are taken on-line; that is, during operation of the hydrocarbon processing environment from which hydrocarbon fluid is to be tested.

For example, during an on-line operation of the hydrocarbon processing environment, the first sensor 12 and the second sensor 14 are disposed in series along the first line 18, and are maintained at relatively higher and lower temperatures T_{Lo} and T_{Hi}, respectively, using one or more of the methods described above. The first and second sensors 12, 14, preferably simultaneously, measure density of the fluid as it passes through the sensors along the first line 18. On-line density measurements D_{TLo}, D_{THi} from the first and second sensors 12, 14 are received by the processor 34 via the signal lines 36. “Simultaneously” refers to the two density measurements being taken within zero to ten seconds of one another, and more preferably between zero and five seconds of each other.

In a particular example method, density and temperature measurements, along with other measurements such as viscosity and dielectric measurements, are taken from the first and second sensors 12, 14 (e.g., fluid properties sensors) and from the temperature sensors 50 disposed for measuring temperatures for each of the first and second density sensors, and these measurements are sent (e.g., transmitted) to the processor 34. Alternatively, if the first and second temperatures are already known (for example, they are maintained independently, but at temperatures known by the processor 34), the processor 34 may only receive the density measurements. These measurements may be stored in memory or recorded, e.g., stored in one or more databases associated with (e.g., in communication with) the processor 34. Each sensor output may be tagged by the processor 34. An example sampling rate for receiving measurements can be, for instance, every 0.1 to 10 seconds, and more preferably, every 0.5 to 5 seconds, though samples can be taken at any desired time(s) or periods.

In step 54, the processor 34 determines, e.g., calculates, a temperature conversion factor, or a coefficient of thermal expansion γ, from D_{THi}, D_{TLo}, T_{Hi}, and T_{Lo}, by using the difference in the densities D_{TLo}, D_{THi} and the difference in the temperatures T_{Lo} and T_{Hi}. For example, the coefficient of thermal expansion γ can be calculated using equation (2) below:

$$γ = \frac{( {D}_{THi} - {D}_{TLo} )}{( {T}_{Hi} - {T}_{Lo} )} \qquad ( 2 )$$

Alternatively, the processor 34 can calculate a temperature conversion factor such as coefficient B in Equation (1) below:

$${D}_{1} = \frac{{D}_{0}}{( 1 + B ( {T}_{1} - {T}_{0} ) )} \qquad ( 1 )$$

by inputting one of the first and second densities (e.g., either D_{THi} or D_{TLo}) measured in step 52 for D₀, inputting the other measured density for D₁), inputting the associated (i.e., high or low) temperature T_{Hi}, and T_{Lo}, for T₀ and T₁ (e.g., if D_{THi} is input as D₁, then T_{Hi} would be input as T₁, and D_{TLo} and T_{Lo} would be input for D₀ and T₀, respectively), and solving for the temperature conversion factor B.

Next, in step 56, the corrected measured density is determined using measured density and the temperature conversion factor. Particularly, the processor 34 corrects the measured density for temperature from either the first density sensor 12 or the second density sensor 14 using the determined temperature conversion factor. This can be performed by, for example, converting the measured density from either the first density sensor 12 or the second density sensor 14 to standard temperature using the calculated temperature conversion factor, e.g., the coefficient of thermal expansion γ (using equation (2)), or the temperature conversion factor B (using equation (1)). For instance, for a standard temperature of 15.55° C. (60° F.), the density D_{TLo} measured at the first (lower temperature sensor) 12 at temperature T_{Lo} can be converted to standard density D_{60F} using equation (3) below, which can be derived from equation (2):

D_{60F}=D_{TLo}+γ*(T_{Lo}−60 F) (3)

In step 58, the resulting corrected density, e.g., standard density D_{60F}, is output. For example, the corrected density can be output to a plant or laboratory data system, printed, displayed on a suitable display, stored within memory, stored within non-volatile storage, output for further processing compared to a threshold to trigger an alarm condition, etc.

In a particular example, the processor further may calculate the specific gravity of the fluid by dividing the standard density at, say, 15.55° C. (60° F.), by density of water at 60° F., 0.99907 g/ml. In this way, corrections for variation of liquid being measured are incorporated into temperature-adjusted specific gravity measurement.

Similarly, the processor 34 further may calculate the API gravity of the fluid by using equation (4), below

$$API = \frac{141.5}{Specific Gravity of fluid at 15.55 ° C .} - 131.5 \qquad ( 4 )$$

Thus, example on-line measurement of API gravity allows conversion data to be calculated in real-time.

Example methods provide on-line density measurement for hydrocarbon fluids in various hydrocarbon processing environments, while correcting for variations in fluid conditions during operation. More laboratory workload can be moved on-line. Example on-line density measurement methods provide tighter plant control, reduce fluid testing cost, and increase experimental throughput by minimizing plant line out time after conversion and distillation set point changes.

Example hydrocarbon fluids to be tested include pure and mixtures of pure liquid hydrocarbon compounds; hydrocracking and hydrotreating process liquid feed, intermediate and final product materials.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

### Specific Embodiments

While the following is described in conjunction with specific embodiments, it will be understood that this description is intended to illustrate and not limit the scope of the preceding description and the appended claims.

A first embodiment of the invention is a method for on-line density measurement for a hydrocarbon fluid, the method comprising causing the hydrocarbon fluid to flow through first and second density sensors arranged in series, the first density sensor having a first temperature and the second density sensor having a second temperature, wherein a temperature difference of between 5° C. and 100° C. is defined between the first temperature and the second temperature; receiving a first density measurement from the first density sensor; receiving a second density measurement from the second density sensor; determining a temperature conversion factor using the first density measurement, the second density measurement, the first temperature, and the second temperature; and correcting either the first density measurement or the second density measurement using the determined temperature correction factor to provide a temperature-corrected density measurement for the hydrocarbon fluid. An embodiment of the invention is one, any or all of prior embodiments in this paragraph up through the first embodiment in this paragraph, wherein the first temperature is lower than the second temperature. An embodiment of the invention is one, any or all of prior embodiments in this paragraph up through the first embodiment in this paragraph, wherein the second temperature is lower than the first temperature. An embodiment of the invention is one, any or all of prior embodiments in this paragraph up through the first embodiment in this paragraph, further comprising during the causing the hydrocarbon fluid to flow, maintaining the first density sensor at the first temperature and the second density sensor at the second temperature. An embodiment of the invention is one, any or all of prior embodiments in this paragraph up through the first embodiment in this paragraph, wherein the maintaining the first density sensor at the first temperature and the second density sensor at the second temperature comprises maintaining the first temperature within a first temperature zone, wherein the first density sensor is disposed within the first temperature zone; and maintaining the second temperature within a second temperature zone, wherein the second density sensor is disposed within the second temperature zone. An embodiment of the invention is one, any or all of prior embodiments in this paragraph up through the first embodiment in this paragraph, wherein the maintaining the first temperature within the first temperature zone comprises one or more of insulating the first temperature zone, selectively heating the first temperature zone, or selectively cooling the first temperature zone; and wherein the maintaining the second temperature within the second temperature zone comprises one or more of insulating the second temperature zone, selectively heating the second temperature zone, or selectively cooling the second temperature zone. An embodiment of the invention is one, any or all of prior embodiments in this paragraph up through the first embodiment in this paragraph, wherein the second temperature zone is disposed within the first temperature zone. An embodiment of the invention is one, any or all of prior embodiments in this paragraph up through the first embodiment in this paragraph, wherein the maintaining the first density sensor at the first temperature and the second density sensor at the second temperature further comprises insulating the first temperature zone; and selectively heating the second temperature zone to the second temperature. An embodiment of the invention is one, any or all of prior embodiments in this paragraph up through the first embodiment in this paragraph, wherein the determining a temperature correction factor comprises determining a temperature correction coefficient. An embodiment of the invention is one, any or all of prior embodiments in this paragraph up through the first embodiment in this paragraph, further comprising determining a specific gravity of the hydrocarbon fluid using the corrected first density measurement or second density measurement. An embodiment of the invention is one, any or all of prior embodiments in this paragraph up through the first embodiment in this paragraph, further comprising determining an API gravity of the hydrocarbon fluid using the corrected first density measurement or second density measurement. An embodiment of the invention is one, any or all of prior embodiments in this paragraph up through the first embodiment in this paragraph, further comprising outputting the corrected first density measurement or second density measurement. An embodiment of the invention is one, any or all of prior embodiments in this paragraph up through the first embodiment in this paragraph, wherein the first and second sensors are disposed in series along a fluid line installed within a fluid line of a hydrocarbon processing system. An embodiment of the invention is one, any or all of prior embodiments in this paragraph up through the first embodiment in this paragraph, wherein the hydrocarbon processing system comprises a hydrocarbon processing plant or a laboratory, and wherein the fluid line comprises one or more of a vacuum column overhead stream, a vacuum column bottoms stream, an atmospheric column overhead stream, or atmospheric column bottoms stream.

A second embodiment of the invention is a method for on-line density measurement for a hydrocarbon fluid in a hydrocarbon processing system, the method comprising causing the hydrocarbon fluid to flow through first and second density sensors in series along a fluid line installed within the hydrocarbon processing environment; maintaining the first density sensor at a first temperature and maintaining the second density sensor at a second temperature, wherein a temperature difference of between 5° C. and 100° C. is defined between the first temperature and the second temperature; during operation of the hydrocarbon processing environment, receiving a first density measurement from the first density sensor and receiving a second density measurement from the second density sensor; determining a temperature conversion factor using the first density measurement, the second density measurement, the first temperature, and the second temperature; correcting either the first density measurement or the second density measurement using the determined temperature conversion factor to provide a temperature-corrected density measurement for the hydrocarbon fluid; and outputting the corrected first density measurement or the second density measurement.

A third embodiment of the invention is a density measurement apparatus for on-line density measurement for a hydrocarbon fluid, the apparatus comprising an inlet and an outlet; a fluid line disposed between the inlet and the outlet; first and second density sensors arranged in series along the fluid line; a heater for selectively heating one or more of the first density sensor or the second density sensor to define a temperature difference of between 5° C. and 100° C. between the first density sensor and the second density sensor; and a processor coupled to the first and second density sensors, the processor being configured to receive a first density measurement from the first density sensor and a second density measurement from the second density sensor, wherein the first density sensor has a first temperature and the second density sensor has a second temperature; determine a temperature conversion factor using the first density measurement, the second density measurement, the first temperature, and the second temperature; and correct either the first density measurement or the second density measurement using the determined temperature conversion factor to provide a temperature-corrected density measurement for the hydrocarbon fluid. An embodiment of the invention is one, any or all of prior embodiments in this paragraph up through the third embodiment in this paragraph, wherein the processor is further configured to maintain the first density sensor at the first temperature and the second density sensor at the second temperature. An embodiment of the invention is one, any or all of prior embodiments in this paragraph up through the third embodiment in this paragraph, further comprising an additional fluid line disposed between the inlet and outlet, the additional fluid line bypassing the first and second density sensors; a valve disposed within the fluid line; and a valve disposed within the additional fluid line. An embodiment of the invention is one, any or all of prior embodiments in this paragraph up through the third embodiment in this paragraph, wherein the fluid line and the additional fluid line are insulated to define a first temperature zone; and wherein the second density sensor is disposed within a second temperature zone, the second temperature zone being insulated from the first temperature zone; wherein the heater is configured to selectively heat the second temperature zone. An embodiment of the invention is one, any or all of prior embodiments in this paragraph up through the third embodiment in this paragraph, wherein the first and second density sensors each comprise fluid properties sensors.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A method for on-line density measurement for a hydrocarbon fluid, the method comprising:
causing the hydrocarbon fluid to flow through first and second density sensors arranged in series, the first density sensor having a first temperature and the second density sensor having a second temperature, wherein a temperature difference of between 5° C. and 100° C. is defined between the first temperature and the second temperature;
receiving a first density measurement from the first density sensor;
receiving a second density measurement from the second density sensor;
determining a temperature conversion factor using the first density measurement, the second density measurement, the first temperature, and the second temperature; and
correcting either the first density measurement or the second density measurement using the determined temperature correction factor to provide a temperature-corrected density measurement for the hydrocarbon fluid.

2. The method of claim 1, wherein the first temperature is lower than the second temperature.

3. The method of claim 1, wherein the second temperature is lower than the first temperature.

4. The method of claim 1, further comprising during said causing the hydrocarbon fluid to flow, maintaining the first density sensor at the first temperature and the second density sensor at the second temperature.

5. The method of claim 4, wherein said maintaining the first density sensor at the first temperature and the second density sensor at the second temperature comprises:
maintaining the first temperature within a first temperature zone, wherein the first density sensor is disposed within the first temperature zone; and
maintaining the second temperature within a second temperature zone, wherein the second density sensor is disposed within the second temperature zone.

6. The method of claim 5, wherein said maintaining the first temperature within the first temperature zone comprises one or more of insulating the first temperature zone, selectively heating the first temperature zone, or selectively cooling the first temperature zone; and wherein said maintaining the second temperature within the second temperature zone comprises one or more of insulating the second temperature zone, selectively heating the second temperature zone, or selectively cooling the second temperature zone.

7. The method of claim 5, wherein the second temperature zone is disposed within the first temperature zone.

8. The method of claim 7, wherein said maintaining the first density sensor at the first temperature and the second density sensor at the second temperature further comprises insulating the first temperature zone; and selectively heating the second temperature zone to the second temperature.

9. The method of claim 1, wherein said determining a temperature correction factor comprises determining a temperature correction coefficient.

10. The method of claim 1, further comprising determining a specific gravity of the hydrocarbon fluid using said corrected first density measurement or second density measurement.

11. The method of claim 1, further comprising determining an API gravity of the hydrocarbon fluid using said corrected first density measurement or second density measurement.

12. The method of claim 1, further comprising outputting said corrected first density measurement or second density measurement.

13. The method of claim 1, wherein the first and second sensors are disposed in series along a fluid line installed within a fluid line of a hydrocarbon processing system.

14. The method of claim 13, wherein the hydrocarbon processing system comprises a hydrocarbon processing plant or a laboratory, and wherein the fluid line comprises one or more of a vacuum column overhead stream, a vacuum column bottoms stream, an atmospheric column overhead stream, or atmospheric column bottoms stream.

15. A method for on-line density measurement for a hydrocarbon fluid in a hydrocarbon processing system, the method comprising:
causing the hydrocarbon fluid to flow through first and second density sensors in series along a fluid line installed within the hydrocarbon processing environment;
maintaining the first density sensor at a first temperature and maintaining the second density sensor at a second temperature, wherein a temperature difference of between 5° C. and 100° C. is defined between the first temperature and the second temperature;
during operation of the hydrocarbon processing environment, receiving a first density measurement from the first density sensor and receiving a second density measurement from the second density sensor;
determining a temperature conversion factor using the first density measurement, the second density measurement, the first temperature, and the second temperature;
correcting either the first density measurement or the second density measurement using the determined temperature conversion factor to provide a temperature-corrected density measurement for the hydrocarbon fluid; and
outputting the corrected first density measurement or the second density measurement.

16. A density measurement apparatus for on-line density measurement for a hydrocarbon fluid, the apparatus comprising:
an inlet and an outlet;
a fluid line disposed between said inlet and said outlet;
first and second density sensors arranged in series along the fluid line;
a heater for selectively heating one or more of the first density sensor or the second density sensor to define a temperature difference of between 5° C. and 100° C. between the first density sensor and the second density sensor; and
a processor coupled to said first and second density sensors, said processor being configured to:
receive a first density measurement from the first density sensor and a second density measurement from the second density sensor, wherein the first density sensor has a first temperature and the second density sensor has a second temperature;
determine a temperature conversion factor using the first density measurement, the second density measurement, the first temperature, and the second temperature; and
correct either the first density measurement or the second density measurement using the determined temperature conversion factor to provide a temperature-corrected density measurement for the hydrocarbon fluid.

17. The density measurement apparatus of claim 16, wherein said processor is further configured to maintain the first density sensor at the first temperature and the second density sensor at the second temperature.

18. The density measurement apparatus of claim 16, further comprising:
an additional fluid line disposed between said inlet and outlet, said additional fluid line bypassing said first and second density sensors;
a valve disposed within said fluid line; and
a valve disposed within said additional fluid line.

19. The density measurement apparatus of claim 16, wherein said fluid line and said additional fluid line are insulated to define a first temperature zone; and wherein said second density sensor is disposed within a second temperature zone, the second temperature zone being insulated from the first temperature zone; wherein said heater is configured to selectively heat the second temperature zone.

20. The density measurement apparatus of claim 16, wherein said first and second density sensors each comprise fluid properties sensors.
